(19) United States
(12) Patent Application Publication
(10) Pub. No.: US 2013/0261424 A1
(43) Pub. Date: Oct. 3, 2013

(54) System for Inducing Respiration Using Biofeedback Principle

(71) Applicant: RESEARCH & BUSINESS FOUNDATION SUNGKYUNKWAN UNIVERSITY, (US)

(72) Inventors: Young Yih HAN, Seoul (KR); Eun Hyuk SHIN, Seoul (KR); Jong In PARK, Seoul (KR); Hee Chul PARK, Seoul (KR); Jung Suk SHIN, Seoul (KR)

(73) Assignee: RESEARCH & BUSINESS FOUNDATION SUNGKYUNKWAN UNIVERSITY, Suwon-si (KR)

(21) Appl. No.: 13/790,372

(22) Filed: Mar. 8, 2013

(30) Foreign Application Priority Data
Mar. 9, 2012 (KR) 10-2012-0024643
Mar. 6, 2013 (KR) 10-2013-0024184

(51) Int. Cl. A61B 5/00; A61B 5/08; A61N 5/10

(52) U.S. Cl. CPC A61B 5/0082; A61N 5/1048; A61B 5/08
USPC 600/407

(57) ABSTRACT

Disclosed herein is a system for processing a biofeedback-treated, respiration-induced signal image using the biofeedback principle, wherein a process for processing the respiration-induced signal image comprises the steps of: (a) acquiring a respiration-induced signal image from a treatment site of a patient in need of radiotherapy treatment using a photographing device; (b) binarizing the acquired image of step (a) using a threshold value; (c) determining whether the binarized image of step (b) is distorted; (d) synthesizing the image; (e) determining whether the synthesized image is required to be separated into regions; and (f) performing pattern matching for the respiration-induced signal image. According to the system, a patient can see and control the patient's own motion at a place where the patient's steady respiration or motion is required, and thus the stability of the patient in a radiotherapy treatment process can be ensured.

### CROSS REFERENCE TO RELATED APPLICATIONS

This application claims the benefit of Korean Patent Application Nos. 10-2012-0024643 filed on Mar. 9, 2012, and 10-2013-0024184 filed on Mar. 6, 2013, in the Korean Intellectual Property Office, the disclosures of which are incorporated herein by reference in their entirety for all purposes.

### BACKGROUND OF THE INVENTION

1. Field of the Invention

The present invention relates to a system for inducing respiration using the biofeedback principle in radiotherapy treatment, and more particularly to a system for inducing respiration using the biofeedback principle in radiotherapy treatment, wherein the respiratory cycle and pattern of a patient are imaged and processed so that steady respiration is induced using a biofeedback signal.

2. Description of the Prior Art

Importantly, four-dimensional radiotherapy or respiratory-gated radiotherapy for a patient should be performed on the premise that organ motion is steady during the radiotherapy treatment. Various factors may influence the motion of human body organs, and among them, keeping breathing steady is very important in radiation therapy.

In radiotherapy using a medical radiotherapy equipment, it can be most important to focus radiation on a tumor site while minimizing the dose to the surrounding normal tissue. Particularly, radiation therapy for a moving organ essentially requires the technology of controlling beams depending on variations in the geometric motion of organs during the treatment.

Various systems are used to satisfy the respiration-related precondition as described above, but these systems mostly use only guide signals, and thus a patient cannot identify the patient's own respiratory pattern and breathes as trained only.

For this purpose, a gating method of controlling a radiotherapy system using a real-time position management (RPM) system is mainly used in four-dimensional (or respiratory gated radiation therapy) radiation therapy. However, the therapeutic success or failure of this technology depends on the respiratory stability of the patient, and respiratory-gated radiotherapy treatment for a patient who breathes unsteadily requires a long treatment time. In addition, in the RPM system, a respiratory phase determined based on a respiratory cycle only without considering the respiratory pattern or volume is applied to respiratory-gated radiotherapy, and thus the accuracy of treatment is significantly low.

In particular, stereotactic body radiotherapy for a moving organ can require a significantly long treatment time and have a very high risk of complication, compared to other radiotherapy methods. To resolve these problems, training a patient to maintain a steady breathing cycle and volume is of prime importance.

For radiotherapy patients with lung-related diseases, it is very difficult to maintain a steady breathing cycle and volume. Thus, it has been suggested that, when respiratory-dated radiotherapy which is required, it needs to be performed only in a steady respiratory statea respiratory aid system should necessarily be used.

A respiratory training method using biofeedback will be described with reference to prior art patent documents. According to the disclosure of Korean Patent Nos. 10-0601932 and 10-0943180, biological signals are measured, collected and analyzed, and the analyzed data are suitably provided to the patient using the biofeedback system. This process makes the patient's breathing steady or helps the body to relax, so that the patient can control the patient's own breathing.

Meanwhile, in the above-mentioned patent documents, there are limits to providing various image processing methods according to a measurement environment in a process of processing collected biological information using image signals. In other words, the patent documents do not describe a visual tracking algorithm which can be obtained by pre-processing images collected from the human body and suitably recombining the processed images. Thus, there is difficulty in accurately matching the pattern between guided respiration and actual respiration.

### Prior Art Documents

### Patent Documents

Korean Patent No. 10-0601932

Korean Patent No. 10-0943180

### SUMMARY OF THE INVENTION

Accordingly, the present invention has been made in order to solve the above-described problems occurring in the prior art, and it is an object of the present invention to provide a system for inducing respiration using the biofeedback principle, wherein an image collected from the human body in a radiation treatment process is pre-processed using a suitable threshold value, and optimal image tracking is performed depending on an each environment using the pre-processed image.

To achieve the above object, the present invention provides a system for processing a biofeedback-treated, respiration-induced signal image, wherein a process for processing the respiration-induced signal image comprises the steps of: (a) acquiring a respiration-induced signal image from a treatment site of a patient in need of radiotherapy treatment using a photographing device; (b) binarizing the acquired image of step (a) using a threshold value; (c) determining whether the binarized image of step (b) is distorted; (d) synthesizing the image; (e) determining whether the synthesized image is required to be separated into regions; and (f) performing pattern matching for the respiration-induced signal image.

The process for processing the respiration-induced signal image may further comprise step (g) of performing edge detection if it is determined in step (c) that the binarized image is distorted.

A mask which is used for the edge detection of step (g) is preferably one or more selected from the group consisting of Prewitt, Roberts, Sobel, Laplacian, and Canny.

The process for processing the respiration-induced signal image may further comprise, if it is determined in step (e) that the synthesized image is required to be separated into regions, step (h) of setting the region of interest for the respiration-induced signal image, and step (i) determining a template image for the region of interest.

### BRIEF DESCRIPTION OF THE DRAWINGS

FIG. 1 is a schematic view of a visual tracking program which is used in a system for inducing respiration using the biofeedback principle according to the present invention.

FIG. 2 is a schematic view of a biofeedback system employing the individual's respiratory pattern.

FIG. 3 is an algorithm showing a process of matching the pattern between a respiration-induced signal and a biofeedback signal by processing images collected from the inventive system for inducing respiration using the biofeedback principle.

### DETAILED DESCRIPTION OF THE INVENTION

The above and other objects, features and advantages of the present invention will be more apparent from the following preferred embodiments with reference to the accompanying drawings. The embodiments described are for illustrative purposes only and are not intended to limit the scope of the present invention.

Hereinafter, a system for inducing respiration using the biofeedback principle in radiotherapy treatment according to an embodiment of the present invention will be described with reference to the accompanying drawings.

Configuration of System for Inducing Respiration Using Biofeedback Principle

The inventive system for inducing respiration using biofeedback principle makes it possible to construct a patient training system for inducing steady and regular respiration using the biofeedback principle in simulated radiotherapy treatment or actual radiotherapy treatment or to monitor real-time respiration using visual tracking and image processing.

According to the present invention, a biofeedback signal is displayed to the patient using a visual device so that the steady and regular respiration of the patient can be induced. Alternatively, the system of the present invention is used to control a radiation treatment device or to generate alarm.

In the present invention, a respiration-induced signal can be provided as a conventional sine-type signal or a patient-tailored signal according to each respiration model.

In the present invention, an image acquired in real time from a patient is analyzed and displayed, so that a real-time technology of less than 50 msec, preferably less than 30 msec, can be realized while actual circumstances are displayed for effective determination.

In addition, using an ROI (region of interest) setting method for effective tracking analysis, the speed of real-time technology is optimized. Also, using a PID automatic control method, a technology of automatically controlling a treatment device according to an increase in irregularity is realized while alarm is generated.

In addition, time lag is given between a respiration-induced signal and a biofeedback signal to induce the most suitable respiration, and the respiration can be confirmed through a real-time biofeedback signal.

Additionally, the present invention is not limited to the use of an image for receiving and analyzing a respiratory signal and encompasses other recognition methods capable of receiving the respiratory signal.

Hereinafter, an image tracking program and an image processing and pattern matching process, which can embody the above-described features of the present invention, will be described.

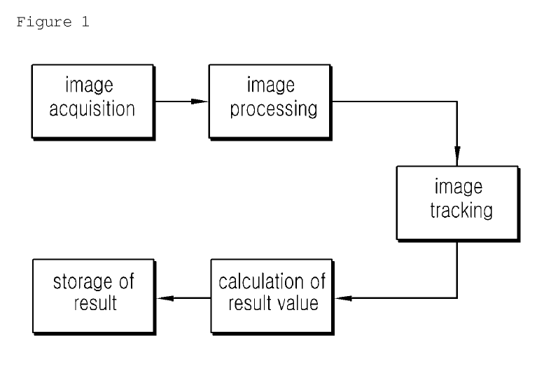

FIG. 1 shows a visual tracking program which is used in a system for inducing respiration using the biofeedback principle according to the present invention.

As shown in FIG. 1, the visual tracking program that is used in the present invention acquires an image from the treatment site of a patient and processes the acquired image. After image processing, visual tracking is performed for precise comparative analysis with the object to be identified. After the visual tracking has been completed, the result value is calculated and stored.

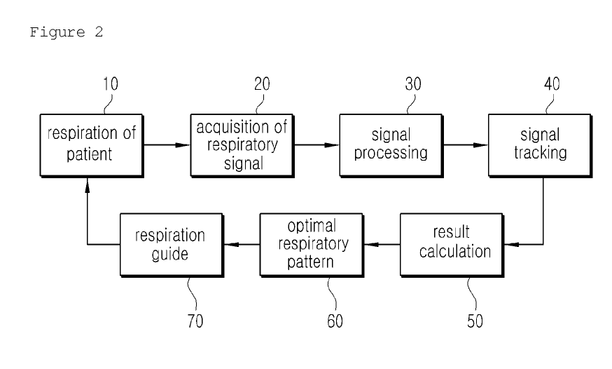

FIG. 2 shows a biofeedback system that uses the individual's respiratory pattern to induce regular respiration.

As shown in FIG. 2, a patient who breathes unsteadily is selected and allowed to breathe naturally (10), and a respiratory signal generated in the breathing procedure is acquired (20). The acquired respiratory signal is processed so as to be smoothly tracked (30), and signal tracking is performed based on the treated signal (40), after which the result is obtained based on the tracked signal (50). Based on the obtained result, the optimum respiratory pattern is found (60), and the obtained respiratory pattern is guided to the patient who breathes unsteadily (70).

FIGS. 1 and 2 allow steady and regular respiration to be induced during radiotherapy treatment by a series of procedures using the biofeedback principle.

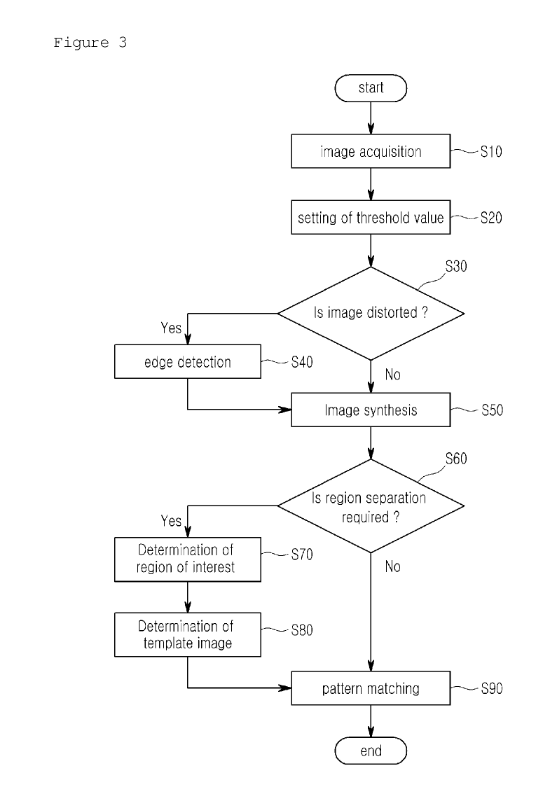

FIG. 3 is a time sequential flow chart showing an image processing and a pattern matching process which is used in the present invention.

As shown in FIG. 3, an image is acquired from the treatment site of a patient, who is undergoing radiotherapy, using a photographing device such as a CCD or an IR camera (S10).

After step S10, the image is pre-processed using a binarization method or a threshold value in order to increase the accuracy of visual tracking technology (S20). In one example of the present invention, according to a binarization method, the acquired image is converted into a binary image having digital values of ‘0’ and ‘1’, and a specific threshold value is determined. If the pixel value of the acquired image is greater than the threshold value, it is expressed as white, and if the pixel value of the acquired image is smaller than the threshold value, it is expressed as black.

After binarizing the acquired image using the threshold value in step S20, whether the obtained image is distorted is determined (S30). In step S30, if the image includes a region having a relatively low visual image quality, or has overlapped regions or significantly deviates from the basic shape of the object to be measured, the image can be determined to be distorted.

If it is determined in step S30 that the image is determined to be distorted, the image can be subjected to edge detection (S40). In the edge detection, an edge, which appears in the distorted image region and is not a boundary, is independently detected to prevent the decrease in image quality caused by the application of a high-frequency enhancement filter and to highlight the boundary of lines, thereby obtaining a final image having improved quality (S40). The edge detection is performed by finding the edge using a change in brightness by a differential operator, and various algorithms are used in the edge detection. In addition, the edge detection may also be performed by determining a differential value using partial differential operation. In programming, quick calculation using a mask can be more effective than direct calculation, and the characteristic of the mask is that the sum of all pixels in the mask is 0. The mask that is used in the edge detection may be any one selected from among Prewitt, Roberts, Sobel, Laplacian, Canny and the like.

The undistorted image resulting from step S30 or the image resulting from step S40 is synthesized (S50). In step S50, the image pre-processed in step S20 is processed by various methods selected depending on each environment to make optimal image tracking possible.

After step S50, whether the treatment site of the patient is required to be separated into regions is determined (S60).

If it is determined in step S60 that the region separation is required, the region of interest (ROI) is set to optimize the speed of real-time technology to thereby make effective tracking analysis possible. In other words, an image for a specific tumor site in need of radiotherapy treatment can be set as the region of interest (S70).

As the image of a specific human body site is set as the region of interest in step S70, a template image therefor is set (S80). Herein, the template image can be understood as a reference image for the region of interest.

If it is determined in step S60 that the region separation is not required or if the region of interest and the template image are set in steps S70 and S80, pattern matching is performed (S90).

Specifically, using the image synthesized in step S50 or the region of interest and the template image in steps S70 and S80, the similarity between induced respiration in the patient and biofeedback respiration is analyzed and displayed. Through this procedure, the similarity between current respiration and induced respiration can be easily determined.

Meanwhile, the system of the present invention can function as a system for training self-respiration using a mobile device. Specifically, using various mobile devices, including smart phones and PDAs, the mobility of the patient is improved and the patient is trained to self-breathe. In simulated radiation treatment before actual radiation treatment, a patient-tailored respiratory pattern is acquired and periodically displayed, and during a specific period of time between simulated radiation treatment and actual radiation treatment, the regularity of respiration of the patient can be increased.

In addition, according to the present invention, the regularity of motion of the human body can be improved before four-dimensional radiotherapy treatment or during simulated radiotherapy treatment in order to increase computed tomography image quality.

As described above, according to the inventive system for inducing respiration using the biofeedback principle in radiation treatment, the patient can see and control the patient's own motion at a place where the patient's steady respiration or motion is required, and thus the stability of the patient in a radiotherapy treatment process can be ensured. Specifically, the similarity between a respiration-induced signal and a biofeedback signal is analyzed to determine the similarity between respirations in actual treatment and simulated treatment.

Particularly, when it is difficult for the patient to control the patient's own motion, unsteady motion can be controlled by system control or alarm, thereby securing safe treatment.

In addition, according to the present invention, the regularity of motion of the human body can be improved before four-dimensional radiotherapy treatment or during simulated radiotherapy treatment in order to increase computed tomography image quality.

Although the preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A system for processing a biofeedback-treated, respiration-induced signal image, wherein a process for processing the respiration-induced signal image comprises the steps of:
(a) acquiring a respiration-induced signal image from a treatment site of a patient in need of radiotherapy treatment using a photographing device;
(b) binarizing the acquired image of step (a) using a threshold value;
(c) determining whether the binarized image of step (b) is distorted;
(d) synthesizing the image;
(e) determining whether the synthesized image is required to be separated into regions; and
(f) performing pattern matching for the respiration-induced signal image.

2. The system of claim 1, wherein the process for processing the respiration-induced signal image further comprises step (g) of performing edge detection if it is determined in step (c) that the binarized image is distorted.

3. The system of claim 2, wherein a mask which is used for the edge detection of step (g) is one or more selected from the group consisting of Prewitt, Roberts, Sobel, Laplacian, and Canny.

4. The system of claim 1, wherein the process for processing the respiration-induced signal image further comprises, if it is determined in step (e) that the synthesized image is required to be separated into regions, step (h) of setting the region of interest for the respiration-induced signal image, and step (i) determining a template image for the region of interest.
